(19) United States
(12) Patent Application Publication
(10) Pub. No.: US 2022/0142991 A1
(43) Pub. Date: May 12, 2022

(54) METHODS AND COMPOSITIONS FOR TREATMENT OF L YSOSOMAL STORAGE DISORDER

(71) Applicant: CHILDREN'S HOSPITAL MEDICAL CENTER, Cincinnati, OH (US)

(72) Inventor: Dao PAN, Cincinnati, OH (US)

(21) Appl. No.: 17/438,024

(22) PCT Filed: Mar. 13, 2020
(86) PCT No.: PCT/US2020/022761

§ 371 Date: Sep. 10, 2021

Related U.S. Application Data
(60) Provisional application No. 62/818,220, filed on Mar. 14, 2019.

(51) Int. Cl. A61K 31/436; A61P 25/00

(52) U.S. Cl. CPC A61K 31/436; A61P 25/00

(57) ABSTRACT

Methods for improving at least one neurological function in a subject that has or is suspected of having a neurologic lysosomal storage disorder using a rapamycin compound. Also provided herein are treatment of such a neurologic lysosomal storage disorder with the rapamycin compound.

### CROSS REFERENCE TO RELATED APPLICATIONS

This application claims the benefit of U.S. Provisional Application 62/818,220, filed Mar. 14, 2019, the disclosure of which is hereby incorporated by reference in its entirety.

### GOVERNMENT SUPPORT

This invention was made with government support under NS086134 awarded by the National Institutes of Health. The government has certain rights in the invention.

### BACKGROUND OF THE INVENTION

Lysosomal storage disorders (LSD) include about 70 metabolic diseases, more than half of which affect the central nervous system (CNS). Gaucher disease (GD) is an example of a LSD having debilitating neuropathologic and behavioral manifestations. GD is caused by inherited deficiency of lysosomal enzyme acid β-glucosidase (GCase). Loss of GCase leads to accumulation of Glucosylceramide in visceral organs and CNS. Clinically, GD is divided into three types: non-neuronopathic type 1, acute neuronopathic type 2 and chronic neuronopathic type 3. Specifically, type 2 presents early in infancy leading to death by age of 2. Type 3 GD manifests in childhood and is a more slowly progressive disorder. Baris et al., (2014) Pediatr Endocrinol Rev. 12 Suppl 1(0 1), 72-81; Stirnemann et al., (2017) Int J Mol Sci. 18(2):441.

Current treatment modalities for LSDs are somewhat effective to correct systemic manifestations, but fail to treat or improve the CNS pathologies and/or neurological symptoms for this group of disorders. Thus, there is a need to develop new therapeutic approaches for treatment of LSDs with lower mortality and morbidity, and with the capacity to correct CNS deterioration and declining neurological functions.

### SUMMARY OF THE INVENTION

The present disclosure is based, at least in part, on the unexpected discoveries that rapamycin successfully improved neurological functions and prolonged life span of neuronopathic Gaucher disease as observed in a genetic mouse model. These results suggest that rapamycin compounds would be expected to benefit treatment of neurologic lysosomal storage disorders such as neuronopathic Gaucher disease.

Accordingly, one aspect of the present disclosure provides a method of improving at least one neurological function in a subject having or suspected of having a neurologic lysosomal storage disorder (LSD). Also provided herein are methods for treating such a neurologic LSD in a subject in need of the treatment. Any of the methods disclosed here may comprise: administering to a subject in need thereof an effective amount of a rapamycin compound. In some instances, the rapamycin compound may be sirolimus, everolimus, temsirolimus, ridaforolimus, N-dimethylglycinate-rapamycin, 32-deoxo-rapamycin, zotarolimus, acrolimus or pimecrolimus. Alternatively or in addition, the rapamycin compound may be conjugated to a pharmaceutically acceptable polymer. Any of the rapamycin compounds disclosed herein may be formulated in a pharmaceutical composition, which may further comprise a pharmaceutically acceptable carrier.

In some embodiments, the rapamycin compound may be administered to the subject by a parenteral route (see examples provided herein). Alternatively, the rapamycin compound may be administered to the subject orally.

In some embodiments, the neurologic lysosomal storage disease may be Fabry disease, Farber disease, Gangliosidosis GM1, Krabbe disease, Schindler disease, Sandhoff disease, Tay-Sachs, Metachromatic Leukodystrophy, Niemann-Pick disease, Hurler syndrome, Hurler-Scheie syndrome, Hunter syndrome, Sanfilippo A syndrome, Sanfilippo B syndrome, Sanfilippo C syndrome, Sanfilippo D syndrome, Sly Syndrome, Pompe disease, and Gaucher disease. In some examples, the neurologic lysosomal storage disorder may be neuronopathic Gaucher disease (nGD).

In any of the methods disclosed herein, the subject may be a human patient having the neurologic lysosomal storage disorder. In some examples, the human patient may have Type II nGD. In other examples, the human patient may have Type III nGD.

In some examples, the subject can be a human child patient (e.g., younger than 12) having the neurologic lysosomal disorder. In some examples, the subject may have undergone or may be undergoing another therapy for the neurologic lysosomal disorder.

In some embodiments, the rapamycin compound may be administered to any of the subjects disclosed herein at a dose that leads to a serum level of the rapamycin compound ranging from about 5 to about 60 ng/ml (e.g., oral administration of about 0.5-6 mg/m²). In some examples, the rapamycin compound may be administered by a schedule ranging from three times per day to once per week. In some instances, the rapamycin compound is administered to a subject once a day orally. In other examples, the rapamycin compound can be administered to a subject once a day to once a week by intravenous infusion, for example, once per day or once every other day.

Also within the scope of the present disclosure is a rapamycin compound as disclosed herein or a pharmaceutical composition comprising such for use in treating a neurologic LSD as also disclosed herein. Further provided herein are uses of the rapamycin compound for manufacturing a medicament for use in treating the target LSD.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIGS. 1A and 1B is a series of graphs showing the therapeutic effect of rapamycin treatment on lifespan of a genetic model of neuronopathic Gaucher disease (nGD) (type 2/3), the 4L;C* nGD mouse model. FIG. 1A: Kaplan-Meier curves analyzed by log-rank test showing the median survival of rapamycin-treated 4L;C* mice (4L;C* rapa) wherein by injection of rapamycin at 6 mg/kg/2 days starting from 2-weeks of age significantly extended lifespan to 82 days, compared to untreated-4L;C* mice (59 days) and buffer-treated 4L;C* mice (60 days). Mice unaffected by nGD (4L;norm) out lived all other groups. FIG. 1B: relative bodyweight curves as normalized by weight value of each animal at the age of 30 days, where dotted lines indicate the starting ages (52 and 75 days) for bodyweight to show significant difference from normal controls (4L;norm). N for each group is indicated within ( ).

FIGS. 2A and 2B include graphs showing the therapeutic effect of rapamycin treatment on motor functions in mouse model of nGD. FIG. 2A shows results of gait analysis with mice at age of ˜50 days wherein buffer-treated 4L;C* mice and mice unaffected by nGD (4L;norm) were compared to rapamycin-4L;C* mice (i.p. injection of rapamycin at 6 mg/kg/2 days starting from 2-weeks of age). FIG. 2B shows results of hindlimb clasping assessment with mice at age of 50 days wherein buffer-treated 4L;C* mice and mice unaffected by nGD (4L;norm) were compared to rapamycin-4L;C* mice (i.p. injection of rapamycin at 6 mg/kg/2 days starting from 2-weeks of age).

FIGS. 3A and 3B include graphs showing the therapeutic effect of rapamycin treatment on short-term cognitive deficits in mouse models of nGD. FIG. 3A shows results of exploratory (horizontal) activity in mice at age of 53-54 days wherein buffer-treated 4L;C* mice and mice unaffected by nGD (4L;norm) were compared to rapamycin-4L;C* mice (i.p. injection of rapamycin at 6 mg/kg/2 days starting from 2-weeks of age). FIG. 3B shows results of habitual (grooming) activity in mice at age of 53-54 days wherein buffer-treated 4L;C* mice and mice unaffected by nGD (4L;norm) were compared to rapamycin-4L;C* mice (i.p. injection of rapamycin at 6 mg/kg/2 days starting from 2-weeks of age).

FIGS. 4A-4G include images showing that rapamycin ameliorates neuronal degeneration in a mouse model of nGD. FIG. 4A shows results of quantitative analysis in different brain regions for fluoro-Jade C (FJC) staining for degenerating neurons in sagittal cryosections of brain harvested from well-perfused mice at age of Day 55. FIGS. 4B-4F shows representative FJC staining in the regions of thalamus (FIG. 4B), midbrain (FIG. 4C), cortex (FIG. 4D), brain stem (FIG. 4E) and cerebellar region with deep cerebellar nuclei (CBL DCN) (FIG. 4F) wherein buffer-treated 4L;C* mice and mice unaffected by nGD (4L;norm) were compared to rapamycin-4L;C* mice (i.p. injection of rapamycin at 6 mg/kg/2 days starting from 2-weeks of age). FIG. 4G shows results of quantitative analysis of FJC staining among different brain regions in buffer-treated 4L;C* mice.

FIGS. 5A-5C include images and pathological quantification showing that rapamycin ameliorates reactive astrocytosis in a mouse model of nGD. FIG. 5A shows results of quantitative analysis in different brain regions for immunohistochemistry (IHC) analysis with anti-GFAP antibody for activated astrocytes in sagittal cryosections of brain harvested from well-perfused mice at age of Day 55. FIG. 5B shows GFAP positive staining in the cortex region and FIG. 5C shows GFAP positive in the brain stem region of buffer-treated 4L;C* mice, mice unaffected by nGD (4L;norm) and rapamycin-4L;C* mice (i.p. injection of rapamycin at 6 mg/kg/2 days starting from 2-weeks of age) wherein with areas within red-frames of FIG. 5C are magnified in bottom panel.

FIGS. 6A-6I include images showing that rapamycin ameliorates CNS inflammation in the brain and spinal cord in a mouse model of nGD. FIG. 6A shows representative tile scans of sagittal brain sections with CD68+ positive staining in buffer-treated 4L;C* mice, mice unaffected by nGD (4L;norm) and rapamycin-4L;C* mice (i.p. injection of rapamycin at 6 mg/kg/2 days starting from 2-weeks of age). FIGS. 6B-6H shows representative CD68+ positive staining in cross-sections of spinal cord (FIG. 6B), thalamus (FIG. 6C), midbrain (FIG. 6D), cortex (FIG. 6E), brain stem (FIG. 6F) cerebellar region with deep cerebellar nuclei (CBL DCN) (FIG. 6G), and spinal cord (FIG. 6H) wherein buffer-treated 4L;C* mice and mice unaffected by nGD (4L;norm) were compared to rapamycin-4L;C* mice (i.p. injection of rapamycin at 6 mg/kg/2 days starting from 2-weeks of age). FIG. 6I shows results of quantitative analysis of CD68+ positive staining among different brain regions in, rapamycin-treated 4L;C* mice, buffer-treated 4L;C* mice, and untreated 4L;norm mice.

FIGS. 7A and 7B include images showing that rapamycin normalizes LC3B-II levels in the brain of a mouse model of nGD. FIG. 7A shows a western blot analysis probing for LC3B-I and LC3B-II (top panel) and loading control β-actin (bottom panel) in midbrain tissue harvested from well-perfused WT mice, buffer-treated 4L;C* mice, and rapamycin-treated 4L;C* mice at 55 days of age. FIG. 7B shows results of quantitative analysis of normalized amounts of LC3B-II protein levels in midbrain tissues.

FIGS. 8A-8C include images showing that rapamycin normalizes the hyperactive mTORC1 signaling pathway in the brain of a mouse model of nGD. FIG. 8A shows a representative western blot analysis probing Mac 2, a marker for activated microglia/macrophages (top panel), phosphorylated ribosome protein S6 (Ser235/236), a marker for activated mTORC1 signaling, and the loading control β-actin (second from top panel and bottom panel) in midbrain tissue harvested from well-perfused 4L;norm mice, buffer-treated 4L;C* mice, and rapamycin-treated 4L;C* mice at 55 days of age. FIG. 8B shows results of quantitative analysis of normalized amounts of Mac 2 protein levels in midbrain tissues. FIG. 8C shows results of quantitative analysis of normalized amounts of phosphorylated ribosome protein S6 protein levels in midbrain tissues. N=6-9 for each group.

FIG. 9 is a graph showing that abnormally elevated expression of inflammatory mediators in brains of buffer-treated 4L;C* mice (nGD diseased brains) was reduced by rapamycin treatment in rapamycin-treated 4L;C* mice. Midbrain tissues were used from well-perfused mice harvested at 55 days of age.

FIGS. 10A-10D include images showing that rapamycin treatment effectively ameliorates abnormal microglia proliferation and high occurrences of immune cells in the brain of a mouse model of nGD. FIG. 10A shows representative flow cytometry plots after FACS analysis with staining for microglia and subpopulations of leukocytes isolated from brain hemispheres of 4L;norm mice, buffer-treated 4L;C* mice, and rapamycin-treated 4L;C* mice at the age of 55 Days. FIG. 10B shows quantitative analysis of the frequency of microglial cells and different leukocyte populations isolated from brain hemispheres of 4L;norm mice, buffer-treated 4L;C* mice, and rapamycin-treated 4L;C* mice. FIG. 10C shows representative histogram plots of CD11b expression in microglial cells and FIG. 10D shows quantitative analysis of mean florescent intensity (MFI) of CD11b expression in microglial cells isolated from brain hemispheres of 4L;norm mice, buffer-treated 4L;C* mice, and rapamycin-treated 4L;C* mice.

FIGS. 11A-11C include images showing rapamycin significantly reduced leukocyte migration into the brain of nGD mice. To distinguish real migrated leukocytes from resident brain immune cells, GFP+ low-density bone marrow cells were transplanted into busulfan-treated recipient pups (4L;norm and 4L;C* mice) (nBMT) and followed by injection of buffer or rapamycin. FIGS. 11A and 11B show representative flow cytometry plots after FACS analysis with staining for microglia and subpopulations of leukocytes (FIG. 11A) and donor-derived (migrated) GFP+ cells (Table 1 below) harvested from brain hemispheres of 4L;norm mice, buffer-treated 4L;C* mice, and rapamycin-treated 4L;C* mice at the age of 55 Days. FIG. 11B shows the frequency of GFP+ cells among different leukocyte populations in the brain of recipient mice analyzed by FACS. FIG. 11C shows a qualitative analysis of the frequency of donor-derived GFP+ cells among different leukocyte populations that were analyzed by FACS.

FIGS. 12A-12D include images showing that rapamycin reduced brain inflammation by suppressing activation of microglial cells and leukocyte migration into the brain of nGD mice. FIGS. 12A-12D show immunofluorescence staining for migrated cells (GFP+, green) and macrophage/activated microglia (CD68+, red) in the thalamus (FIG. 12A), brain stem (FIG. 12B), cerebellar region with deep cerebellar nuclei (CBL DCN) (FIG. 12C), and midbrain (FIG. 12D) where red square areas are indicative of enlarged images to the right. White arrow shows migrated macrophages with GFP and CD68 double positive, white triangle shows migrated cells with GFP positive only, and hollow triangle shows resident microglia with CD68 positive only.

### DETAILED DESCRIPTION OF THE INVENTION

The present disclosure is based, at least in part, on the discovery that a rapamycin compound successfully improved neurological functions and expanded life span in a mouse model of neuronopathic Gaucher disease, a representative neurologic lysosomal storage disorder (LSD).

Accordingly, provided herein are methods for improving neurological functions associated with a neurologic LSD and/or treating the neurologic LSD in a subject in need of the treatment by administering to the subject an effective amount of a rapamycin compound.

### Rapamycin Compounds

Rapamycin compounds, as described herein, encompass rapamycin (a.k.a., sirolimus), pharmaceutically acceptable salts or esters thereof, analogues thereof (a.k.a., rapalogs), including prodrugs thereof. In some embodiments, the rapamycin compounds are macrolide compounds containing large (14-16-membered) lactone rings and reduced saccharide substituents. Such a rapamycin compound may be a natural product produced by bacteria. In some examples, a rapamycin compound disclosed herein may comprise a core structure of Formula I:

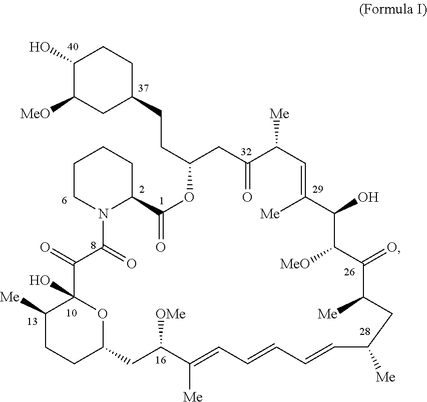 [SMILES: CO[C@H]1C[C@@H]2CC[C@@H](C)[C@@](O)(O2)C(=O)C(=O)N2CCCC[C@H]2C(=O)O[C@H](CC[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

which may optionally be substituted at one or more suitable positions as known to those skilled in the art. Non-limiting examples include positions C16, C32, and/or C40. Suitable substituents include, but are not limited to, C₁₋₃ alkyl, halogen, —CN, —NO₂, —N₃, C₂₋₄ alkenyl, C₂₋₄ alkynyl, —OR, —NH₂, or —SR, R being hydrogen, halogen, —CN, —NO₂, —N₃, acyl, C₁₋₃ alkyl, C₂₋₄ alkenyl, C₂₋₄ alkynyl; and a being 0, 1, 2, 3, 4, or 5.

In some examples, a rapamycin compound disclosed herein may comprise the core structure of Formula I and carry one or more additional functional groups. Non-limiting examples of functional groups include methoxy groups, hydroxyl groups, keto groups, benzene rings, pipecolate rings, cyclohexane rings, amine groups, alcohols, ethers, alkyl halides, thiols, aldehydes, ketones, esters, carboxylic acids, or amides.

Exemplary rapamycin compounds include, but are not limited to, sirolimus, everolimus, temsirolimus, ridaforolimus, N-dimethylglycinate-rapamycin, 32-deoxo-rapamycin, zotarolimus, acrolimus, and pimecrolimus. Additional examples include CCI-779, AP23573, and RAD001. See Tai et al., Pharm Res. 2014, 31(3):706-719, the relevant disclosures of which are incorporated by reference for the purpose and subject matter referenced herein. Further, an exemplary rapamycin prodrug is NSC606698 (e.g., N-dimethylglycinate-methanesulfonic acid salt of rapamycin).

The rapamycin compounds disclosed herein can be synthesized using routine methods. See, e.g., WO 2019/064182 A, the relevant disclosures of which are incorporated by reference for the purpose and subject matter referenced herein.

The rapamycin compounds described herein, where applicable, can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The disclosure additionally encompasses compounds described herein as individual isomers substantially free of other isomers, and alternatively, as mixtures of various isomers.

In some examples, the rapamycin compound used in the methods disclosed herein may be an (R)-isomer. Alternatively, the CTX compound may be an (S)-isomer. In some examples, the rapamycin compound may be a mixture of (R) and (S) isomers.

Any of the rapamycin compounds disclosed herein may be conjugated with a biocompatible polymer, for example, polyethylene glycol (PEG) or copolymer of PEG-poly(lactic acid). Examples include rapamycin-Glyn-Poly[bis(s-Lys)Glut-PEG], in which n is an integer of 1-3, inclusive as disclosed in Tai et al., 2014; or oligo(Lactic Acid)8-Rapamycin Prodrug-Loaded Poly(Ethylene Glycol)-block-Poly(Lactic Acid) (e.g., in micelle form).

The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

### Pharmaceutical Compositions

Any of the rapamycin compounds disclosed herein may be formulated to form a pharmaceutical composition, which further comprises a pharmaceutically acceptable carrier, diluent or excipient. Any of the pharmaceutical compositions to be used in the present methods can comprise pharmaceutically acceptable carriers, excipients, or stabilizers in the form of lyophilized formations or aqueous solutions.

The carrier in the pharmaceutical composition must be “acceptable” in the sense that it is compatible with the active ingredient of the composition, and preferably, capable of stabilizing the active ingredient and not deleterious to the subject to be treated. For example, “pharmaceutically acceptable” may refer to molecular entities and other ingredients of compositions comprising such that are physiologically tolerable and do not typically produce untoward reactions when administered to a mammal (e.g., a human). In some examples, the “pharmaceutically acceptable” carrier used in the pharmaceutical compositions disclosed herein may be those approved by a regulatory agency of the Federal or a state government or listed in the U.S. Pharmacopeia or other generally recognized pharmacopeia for use in mammals, and more particularly in humans.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

In some embodiments, the pharmaceutical compositions or formulations are for parenteral administration, such as intravenous, intra-arterial, intra-muscular, subcutaneous, or intraperitoneal administration. In some embodiments, compositions comprising a rapamycin compound can be formulated for intravenous infusion.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well-known to those skilled in the art.

In some embodiments, the pharmaceutical composition or formulation is suitable for oral, buccal or sublingual administration, such as in the form of tablets, capsules, ovules, elixirs, solutions or suspensions, which may contain flavoring or coloring agents, for immediate-, delayed- or controlled-release applications.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, coloring matter or dyes, with emulsifying and/or suspending agents and with diluents such as water, ethanol, propylene glycol and glycerin, and combinations thereof.

In some embodiments, the pharmaceutical composition or formulation is suitable for intranasal administration or inhalation, such as delivered in the form of a dry powder inhaler or an aerosol spray presentation from a pressurized container, pump, spray or nebulizer with the use of a suitable propellant, e.g., dichlorodifluoromethane, trichlorofluoromethane, dichlorotetrafluoro-ethane, a hydrofluoroalkane, carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g. using a mixture of ethanol and the propellant as the solvent, which may additionally contain a lubricant. Capsules and cartridges (made, for example, from gelatin) for use in an inhaler or insufflator may be formulated to contain a powder mix of the inhibitor and a suitable powder base such as lactose or starch.

The formulations may be presented in unit-dose or multi-dose containers, for example sealed ampoules or vials, and may be stored in a freeze-dried (lyophilized) condition requiring only the addition of the sterile liquid carrier immediately prior to use.

### Therapeutic Applications

In other aspects, the present disclosure provides methods for improving one or more neurological functions in a subject having or suspected of having a neurological LSD or for treating such a neurological LSD using one or more of the rapamycin compounds disclosed herein. To practice the therapeutic methods described herein, an effective amount of a rapamycin compound described herein or a pharmaceutical composition comprising such may be administered to a subject who needs treatment via a suitable route (e.g., intravenous infusion or oral administration of the rapamycin compound). The rapamycin compound may be mixed with a pharmaceutically acceptable carrier to form a pharmaceutical composition (e.g., see disclosures herein) prior to administration, which is also within the scope of the present disclosure.

The subject to be treated by any of the methods disclosure herein may be a mammal (e.g., a human patient or a non-human primate). The subject may have, be suspected of having, or be at risk for a neurologic lysosomal storage disorder. Examples of neurologic LSDs include, but are not limited to, Fabry disease, Farber disease, Krabbe disease, Schindler disease, Sandhoff disease, Tay-Sachs, Metachromatic Leukodystrophy, Niemann-Pick disease, Hurler syndrome, Hurler-Scheie syndrome, Hunter syndrome, Sanfilippo A syndrome, Sanfilippo B syndrome, Sanfilippo C syndrome, Sanfilippo D syndrome, Sly Syndrome, Pompe disease, and Gaucher disease. In some instances, the neurologic lysosomal storage disorder is neuronopathic Gaucher disease (nGD). In other instances, the neurologic lysosomal storage disorder is Type II or Type III nGD.

A subject having a target neurologic LSD can be identified by routine medical examination, e.g., laboratory tests, organ functional tests, CT scans, or ultrasounds. In some embodiments, the subject to be treated by the method described herein may be a human patient who has undergone or is subjecting to a therapy for treating the target LSD.

A subject suspected of having any of such a target LSD might show one or more symptoms of the LSD. A subject at risk for the target LSD can be a subject having one or more of the risk factors for that disorder, for example, carrying a genetic mutation associated with the LSD with no disease manifestations at the time of the treatment.

In some embodiments, the subject may be a human child patient (e.g., a child patient having nGD). Such a child patient have be younger than 16 years. In some examples, a child patient to be treated by the method closed herein may have an age younger than 12, for example, younger than 10, 8, 6, 4 or 2. In some examples, the child patient is an infant, e.g., younger than 12 months. Alternatively, the subject may be a human adolescent patient (e.g., 16-20 years old) or a human adult patient having the neurologic lysosomal disorder.

In some instances, the subject to be treated by the method disclosed herein may have been previously treated for the neurologic lysosomal storage disorder. In other instances, the subject to be treated has a neurologic lysosomal storage disorder and has undergone or is undergoing another therapy for the neurologic lysosomal disorder. Non-limiting examples include enzyme replacement therapy, haematopoietic stem cell (HSC) transplantation, substrate reduction molecule therapy, chaperone therapy, adeno-associated virus gene therapy, HSC-mediated lentiviral vector gene therapy, or the combined therapy disclosed herein. The prior anti-lysosomal storage disorder therapy may be complete. Alternatively, the prior anti-lysosomal storage disorder therapy may still be on-going. In some embodiments, the human patient may exhibit improved systemic manifestations associated with the lysosomal storage disorder (e.g., complete or partial) after the prior therapy.

A pharmaceutical composition comprising any of the rapamycin compounds described herein in an effective amount may be administered by any administration route known in the art, such as parenteral administration, oral administration, buccal administration, sublingual administration, topical administration, or inhalation, in the form of a pharmaceutical formulation comprising the active ingredient, optionally in the form of a non-toxic organic, or inorganic, acid, or base, addition salt, in a pharmaceutically acceptable dosage form. In some embodiments, the administration route is oral administration and the formulation is formulated for oral administration.

“An effective amount” as used herein refers to the amount of each active agent (here the one or more rapamycin compound) required to confer therapeutic effect on the subject, either alone or in combination with one or more other active agents. Effective amounts vary, as recognized by those skilled in the art, depending on route of administration, excipient usage, and co-usage with other active agents. For example, an “effective amount” of a rapamycin compound is the amount of the compound that alone, or together with further doses, produces the desired response, e.g., extend lifespan, delay loss of body weight, improve one or more neurological functions, and/or improve neuropathological manifestations in a subject having a neurological LSD such as nGD. This may involve only slowing the progression of the disease temporarily, although more preferably, it involves halting the progression of the disease permanently. This can be monitored by routine methods or can be monitored according to diagnostic methods of the invention discussed herein. The desired response to treatment of the disease or condition also can be delaying the onset of the disease or condition.

Such amounts will depend, of course, on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons. The exact dosage and schedule may be determined by a physician.

In some embodiments, the amount of a rapamycin compound to be given to a subject may result in a serum level of about 5 to about 60 ng/ml. Such an amount can be determined by those skilled in the art following routine practice. For example, different doses may be given to a subject and the serum level of the compound may be monitored at various time point after the administration to determine the suitable dose that would lead to the target serum level of the compound. In some instances, oral administration of about 0.5-6 mg/m² may be used.

In some examples, one or more rapamycin compounds as disclosed herein may be administered to a subject in need of the treatment in an amount sufficient to improve at least one neurological function, for example, a cognitive memory function, a motor function, or a combination thereof. For example, one or more rapamycin compounds may be given to a subject in an amount to improve the neurological function by at least 10% (e.g., at least 20%, at least 30%, at least 40%, at least 50%, at least 60%, at least 80%, or at least 90%). Non-limiting examples of neurological functions include a motor-function deficit, a cognitive deficit, and a neuropathological defect. In some instances, a motor-function deficit may be gait ataxia. In some instances, a cognitive deficit may be memory loss. In some instances, a neuropathological defect may be neuron degeneration, microglia/macrophage activation, astrogliosis, or a combination thereof. In some instances, a neuropathological defect may be located in the spinal cord, middle brain, brain stem, thalamus, cortex, deep cerebellar nuclei (DCN) region, or a combination thereof.

Alternatively or in addition, one or more rapamycin compounds as disclosed herein may be given to a subject in need of the treatment in an amount sufficient to relay body weight loss and/or expand lifespan.

A rapamycin compound or a pharmaceutical composition comprising such may be given to a patient via various routes, depending upon the dosage, the condition being treated, as well as the purpose it is being used for. For example, the rapamycin compound or the composition may be injected intravenous (intravenous, IV) or by oral administration (e.g., in tablet form), optionally after meals. In some instances, a subject (e.g., a human patient) can be treated by orally administering therapeutically effective doses of rapamycin compound in the range of about 0.5 mg/kg to about 15 mg/kg. In other instances, the rapamycin compound may be given to a subject orally at a dose range of about 0.5-6 mg/m². The rapamycin compound can be repeatedly administered orally as often and as many times as the patient can tolerate until the desired response is achieved. The appropriate oral dose and schedule will vary from patient to patient, but can be determined by the treating physician for a particular patient. In some instances, a rapamycin compound is administered orally by a schedule ranging from three times per day to once per week. In other instances, a rapamycin compound is administered about once per day orally.

Alternatively, a rapamycin compound may be given to a subject by intramuscular injection (IM), or subcutaneous injection, or injection to the abdominal lining (intraperitoneal, IP), or into the lining of the lung (intrapleural). The rapamycin compound may be administered to the subject, once or multiple times, via suitable route, for example, intravenous infusion, at a suitable interval. In some instances, a subject (e.g., a human patient) can be treated by infusing therapeutically effective doses of rapamycin compound in the range of about 0.5 mg to about 15 mg. The infusion can be repeated as often and as many times as the patient can tolerate until the desired response is achieved. The appropriate infusion dose and schedule will vary from patient to patient, but can be determined by the treating physician for a particular patient. In some instances, a subject may be administered the rapamycin compound or a composition comprising such via intravenous infusion once per day to once per week. For example, the subject may be administered the rapamycin compound or the composition comprising such once per day, once every other day, or once per week, via intravenous infusion.

The rapamycin compound described herein may be utilized in conjunction with other types of therapy for lysosomal storage disorder therapy including enzyme replacement therapy, haematopoietic stem cell transplantation, substrate reduction molecule therapy, chaperone therapy, adeno-associated virus gene therapy, HSC-mediated lentiviral vector gene therapy, and so forth. Additional useful agents and therapies can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

Also provided herein are uses of one or more mTOR inhibitors for treating a neurologic LSDs as disclosed herein.

### Kits for Therapeutic Uses

The present disclosure also provides kits for use in treating a neurologic LSD as described herein. A kit for therapeutic use as described herein may include one or more containers comprising a rapamycin compound. The rapamycin compound may be formulated in a pharmaceutical composition.

In some embodiments, the kit can additionally comprise instructions for use of a rapamycin compound in any of the methods described herein. The included instructions may comprise a description of administration of the rapamycin compound or a pharmaceutical composition comprising such to a subject to achieve the intended activity in a subject. The kit may further comprise a description of selecting a subject suitable for treatment based on identifying whether the subject is in need of the treatment. In some embodiments, the instructions comprise a description of administering the rapamycin compound or the pharmaceutical composition comprising such to a subject who has or is suspected of having a neurologic lysosomal storage disorder.

The instructions relating to the use of the rapamycin compound or the pharmaceutical composition comprising such as described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. A rapamycin compound may be considered active agents.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (lRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1. Rapamycin Treatment Extended the Lifespan and Delayed Loss of Body Weight in a Murine Model of Neuronopathic Gaucher Disease (nGD)

The 4L;C* mouse is a transgenic animal model that is viable and mimics neurological deficits analogous to subacute neuronopathic Gaucher disease (nGD). The 4L;C* mice used in this and other examples disclosed herein were generated as described in Sun et al., Hum Mol Genet. 2010 Mar. 15; 19(6): 1088-1097, the disclosure of which is incorporated herein in its entirety. In brief, saposin C deficient (C^{-/-}) mice were generated by introducing a Cys→Pro substitution in the saposin C region of prosaposin. Substitution of this conserved cysteine (Cys) breaks one of the three disulfide bridges of saposin C and resulted in the specific deficiency of saposin C. Saposin C is an essential activator for GCase. GCase V394L (4L/4L) mice were generated by germ-line transmission of a missense point mutation in the GCase (GBA) locus encoding for V394L. Next, saposin C^{-/-} mice were cross-bred with point mutated GCase V394L homozygotes (4L/4L) to generate 4L/wt;C^{-/+} mice. 4L;C* mice (4L/4L;C^{-/-}) were produced by intercrossing 4L/4L;C^{+/-} with 4L/4L;C^{+/-}. The strain background of 4L;C* was originally C57BL/6J/129SvEV and backcrossed into pure C57BL/6J strain for more than 12 generations.

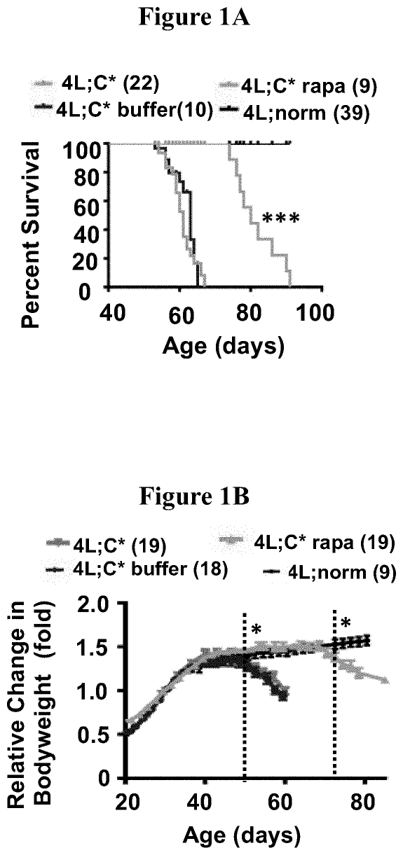

To assess the effect of rapamycin on nGD, rapamycin (6 mg/kg) was administered to 4L;C* mice by intraperitoneal (i.p.) injection every 2 days starting from 2-weeks of age (4L;C* rapa). As a control groups: (1) 4L;C* mice were injected with a buffer which does not contain rapamycin (4L;C* buffer); (2) 4L;C* mice were untreated for the duration of the study (4L;C*); and (3) 4L/4L littermate mice (4L;norm), which have ˜10-20% endogenous GCase activity in different organs and no apparent CNS abnormalities, were used as normal control animals. The lifespan of the mice was monitored daily from 40 days of age until 90 days of age or until death. The Kaplan-Meier method was used to estimate survival probability and a log-rank test was used to compare the Kaplan-Meier survival curves of the four different experimental groups. The median survival of rapamycin-treated 4L;C* mice was significantly extended to 82 days, compared to untreated 4L;C* mice (59 days) and buffer-treated 4L;C* mice (60 days). FIG. 1A shows that rapamycin treatment of 4L;C* mice significantly expanded the lifespan (from 59 days to 82 days), suggesting rapamycin may extend life expectancy for those diagnosed with nGD.

Additionally, the bodyweight the mice was measured daily from 20 days of age until 90 days of age or until death. Relative bodyweight curves were normalized by weight value of each animal at the age of 30 days. At Day 52, there was a significant decline in the relative bodyweight of untreated 4L;C* mice and buffer-treated 4L;C* mice compared to the relative bodyweight of rapamycin-treated 4L;C* mice and 4L/4L control mice. At Day 75, there was a significant decline in the relative bodyweight of rapamycin-treated 4L;C* mice compared to the relative bodyweight of rapamycin-treated 4L;C* mice and 4L/4L control mice. FIG. 1B shows that rapamycin treatment of 4L;C* mice significantly delayed bodyweight decline (from Day 52 days to Day 75) as compared to untreated or buffer-treated 4L;C* controls.

### Example 2. Rapamycin Treatment Improved Neurological Functions in Murine Model of a Neuronopathic Gaucher Disease (nGD)

To determine if administration of rapamycin treatment improves neurological functions in murine model of neuronopathic Gaucher disease (nGD), the potential for neurologic symptomatic improvement was examined using three behavior tests, including gait analysis, hindlimb clasping test and repeated open-field test.

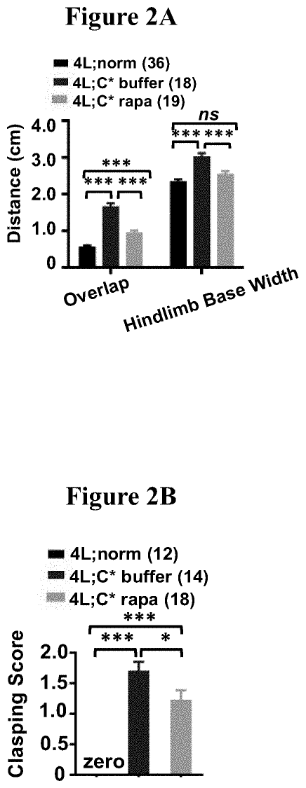

Gait analysis was performed using a paw print test similar to that described in Carter et al., (2001) CURR PROTOC NEUROSCI. Chapter 8: unit 8 12. Briefly, a mouse's front- and hindpaws painted with different colors of water-soluble non-toxic paint and the moue was allowed to walk along a narrow, paper-covered corridor, leaving a track of footprints. Once the footprints dried, the footprints of each walk were analyzed for stride length (left), base lengths, and distance of overlap of the paws. Finally, four parameters were measured from the footprint pattern to describe the locomotion of the mouse: front-paw and hind-paw base width, stride between right front-paw footprints, and overlap (distance between the middle of left high-paw and left front-paw). The first, stride length, was measured as the average distance of forward movement between each stride. The second, hind-base width, was measured as the average distance between left and right hind footprints. This value was determined by measuring the perpendicular distance of a given step to a line connecting its opposite preceding and succeeding steps. The third, front-base width, was measured as the average distance between left and right front footprints. The fourth, overlap between forepaw and hindpaw placement, was measured as the distance between the front and hind footprints on each side. This was used as a measure of the accuracy of foot placement and the uniformity of step alternation. During normal locomotion in rats and mice, the center of the hind footprint falls on top of the center of the preceding front footprint, so that the overlap value is close to zero. With increasing impairment, the footprint placements become more variable and the distance between front and hind prints on the same side increases. Thus, the overlap—the distance between the center of the footprints—becomes greater with increasing impairment. Similar to motor function deficits found in nGD patients with bone infarcts and malformations type 3 ataxia, 4L;C* mice exhibited duck-like waddling due to the splaying of their hindlimbs, and apparent stiffness and bradykinesia (video 1 not shown). FIG. 2A. Rapamycin-treated 4L;C* showed markedly improved movement pattern and locomotor activity toward normal behaviors (video not shown). Gait analysis further verified that rapamycin treatment achieved normalization of hindlimb base width and significant reduction of overlap (FIG. 2A), demonstrating improvement of sensorimotor function.

Next, hind limb clasping was assessed. Briefly, 50-day old mice were suspended by their tail and the extent of hindlimb clasping was observed for 30 seconds. If both hindlimbs were splayed outward away from the abdomen with splayed toes, a score of 0 was given. If one hindlimb was retracted or both hindlimbs were partially retracted toward the abdomen without touching it and the toes were splayed, a score of 1 was assigned. If both hindlimbs were partially retracted toward the abdomen and were touching the abdomen without touching each other, a score of 2 was given. If both hindlimbs were fully clasped and touching the abdomen, a score of 3 was assigned. As shown in FIG. 2B, 4L;C* mice treated with rapamycin showed significantly decreased hindlimb clasping score, suggesting improved motor function of hindlimb toward normal at 50 days of age.

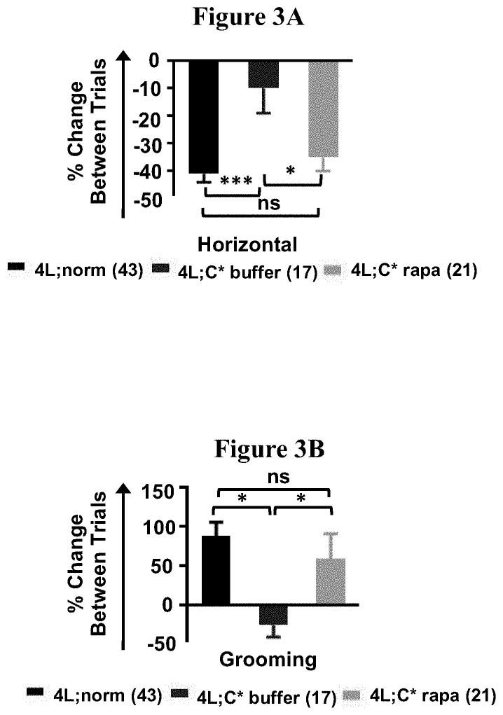

Finally, repeated open-field test was conducted with mice at 53-54 days of age to evaluate exploratory behavior and short-term memory in new environment. The open-field apparatus (60×60 cm) consisted of a white Plexiglas box with 25 squares (12×12 cm) painted on the floor (16 outer and 9 inner). Briefly, each mouse was placed in one of the four corners of the apparatus and allowed to explore for 5 minutes. Activity was monitored and quantified for ambulation (number of squares crossed) of exploratory (horizontal) activity and time spent grooming by two observers in blinded experiments. Each mouse was tested for three repeated trials with 30-minute inter-trial intervals. FIGS. 3A and 3B show normalization of exploratory (horizontal) activity and habitual (grooming) activity in rapamycin treated 4L;C* mice compared to 4L;C* buffer-treated mice. These data indicate that correction of the short-term cognitive deficits could be achieved by rapamycin treatment.

### Example 3. Rapamycin Treatment Improved Neuropathological Manifestations of Neuronopathic Gaucher Disease in a nGD Murine Model

Considering the neurobehavioral improvement could be related with ameliorated brain pathology, the effect of rapamycin on neuropathological manifestations was investigated. The nGD can be characterized by severe neuronal loss, microglial activation, and astrogliosis both in patients and the nGD mouse model. Burrow, et al., (2015) Mol. Genet. Metab., 114 (2), 233-241; Wong et al., (2004) Mol. Genet. Metab., 82 (3), 192-207; Enquis et al., (2007) PNAS, 104 (44) 17483-17488; Sun et al (2010), Hum. Mol. Genet., 19(6), 1088-1097. Fluoro Jade C (FJC), Glial fibrillary acidic protein (GFAP), and CD68 staining were performed to evaluate the effect of rapamycin on neurons, astrocytes, and activated microglia/macrophage respectively.

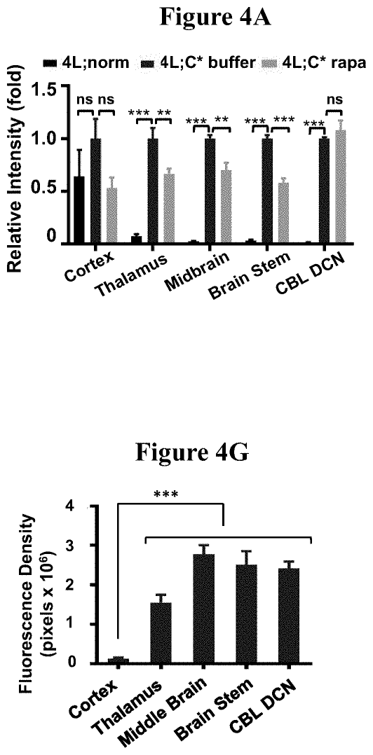

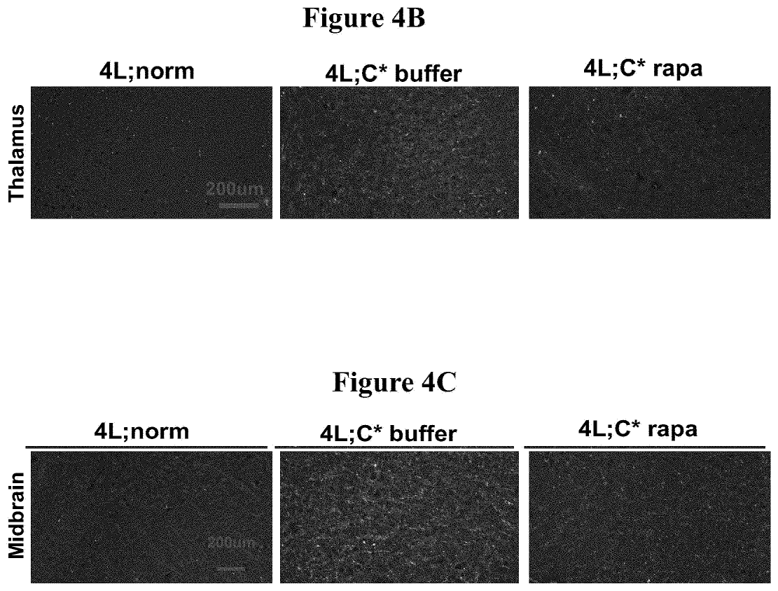

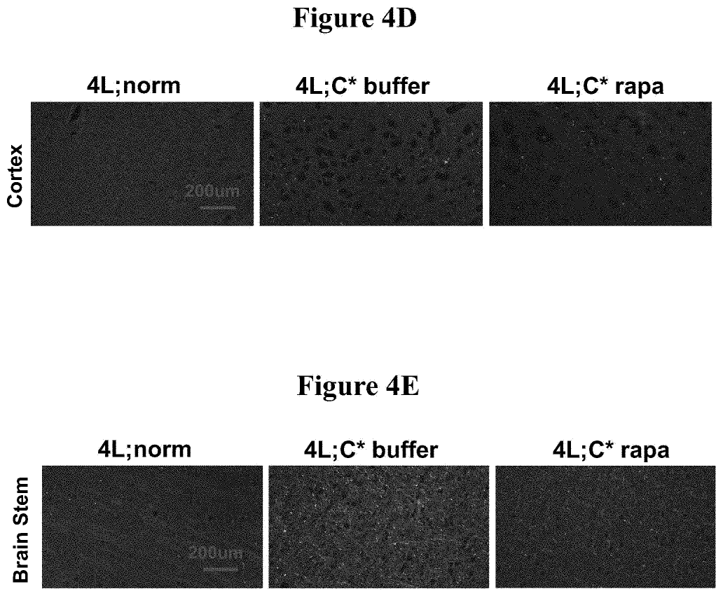

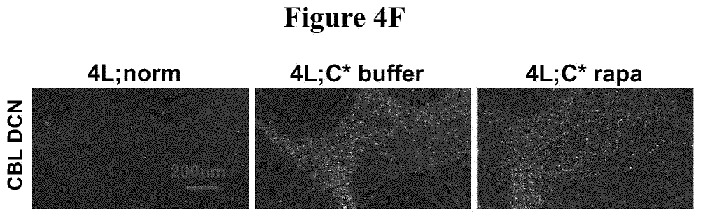

Neuropathological studies have shown that FJC, a polyanionic fluorescein derivative dye, can sensitively and selectively bind to degenerative neurons. To visualize degenerative neurons in nGD mice, brains were harvested from well-perfused rapamycin treated 4L;C* mice, buffer-treated 4L;C* mice, and 4L normal, untreated mice at age of Day 55. Sagittal brain cryosections (10 μm) were mounted on gelatin-coated slides, air-dried, and subjected to FJC staining. The slides were first immersed in a solution containing 1% NaOH in 80% ethanol for 5 minutes. They were rinsed for 2 minutes in 70% ethanol and for 2 minutes in distilled water, then incubated in 0.06% potassium permanganate solution for 10 minutes. Following a water rinse for 2 minutes, slides were transferred to the FJC staining solution and stained for 10 minutes. The proper dilution was accomplished by first making a 0.01% stock solution of FJC dye (Chemicon, Temecula, Calif., USA) in distilled water and then adding 1 ml of the stock solution to 99 ml of 0.1% acetic acid. Slides were washed three times each for 1 minute and then air-dried on a slide warmer at 50° C. for 30 minutes. After clearing in xylene and coverslipping, sections were examined under an epifluorescence microscope. The fluorescein/FITC filter system was used for visualizing FJC staining and images were captured for demonstration. FJC staining revealed that 4L;C* buffer mice showed strong neuronal degenerating signals in midbrain, brain stem and deep cerebellar nuclei (DCN) regions followed by thalamus, with minimal signals observed in cortex. FIGS. 4A to 4G. Rapamycin treatment significantly reduced the degenerating neuronal signals (by 29-45%) in most brain regions (FIG. 4A), except minor change in cerebellar DCN as shown in FIG. 4F. These results indicated that rapamycin significantly ameliorates neuron degeneration in 4L;C* brain, which could translate to improved cognitive and sensorimotor activity in behavior test.

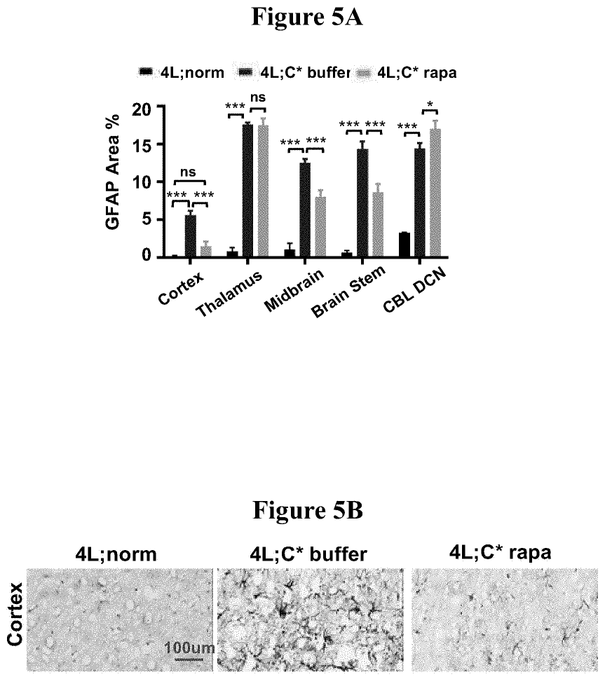

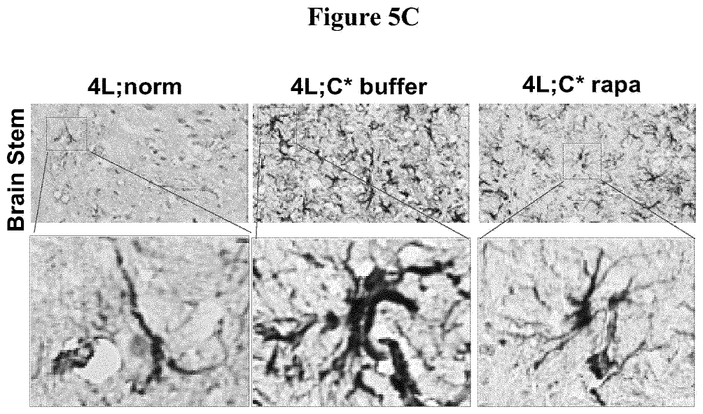

GFAP expression is a sensitive and reliable marker that labels reactive astrocytes responding to CNS injuries, a pathological hallmark of CNS lesions in nGD patients. Sofroniew (2014), Cold Spring Harb Perspect Biol. 7(2):a020420. To visualize reactive astrocytes in nGD mice, immunohistochemistry (IHC) was performed. In brief, brains were harvested from well-perfused rapamycin treated 4L;C* mice, buffer-treated 4L;C* mice, and 4L normal mice at age of Day 55. Sagittal brain cryosections (8 um) were mounted on gelatin-coated slides, air-dried, and subjected to IHC staining using the BenchMark XT IHC/ISH staining module. For GFAP staining, the sections were incubated in buffer solution containing 0.1% trypsin for 5 minutes at room temperature. For morphometric analysis, hematoxylin-eosin co-staining was performed according to standard protocols. Endogenous peroxidase activity was quenched by incubation of the sections for 5 minutes with 3% hydrogen peroxide. Sections were incubated overnight at 4° C. in primary antibodies for GFAP rabbit polyclonal antibody. Staining was detected using biotin-labeled anti-rabbit secondary antibodies and streptavidin conjugated to horseradish peroxidase. Sections were examined with microscope using computer-assisted image analysis software. After rapamycin treatment, 4L;C* mice showed reduced numbers of activated astrocytes, decreased expression of GFAP and thin cell body, especially in the cortex and brain stem regions. FIGS. 5A-5C. Intriguingly, the GFAP signal was decreased (by 36-74%) in middle brain, brains stem and even normalized in cortex, except no reduction in thalamus and cerebellar DCN. FIG. 5A.

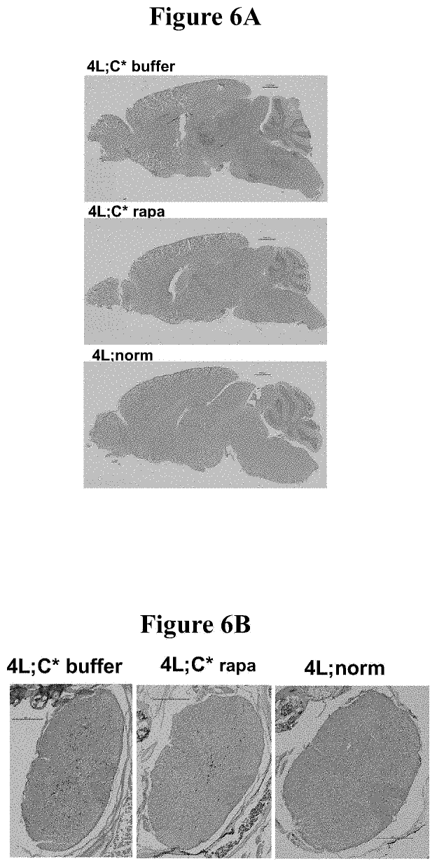

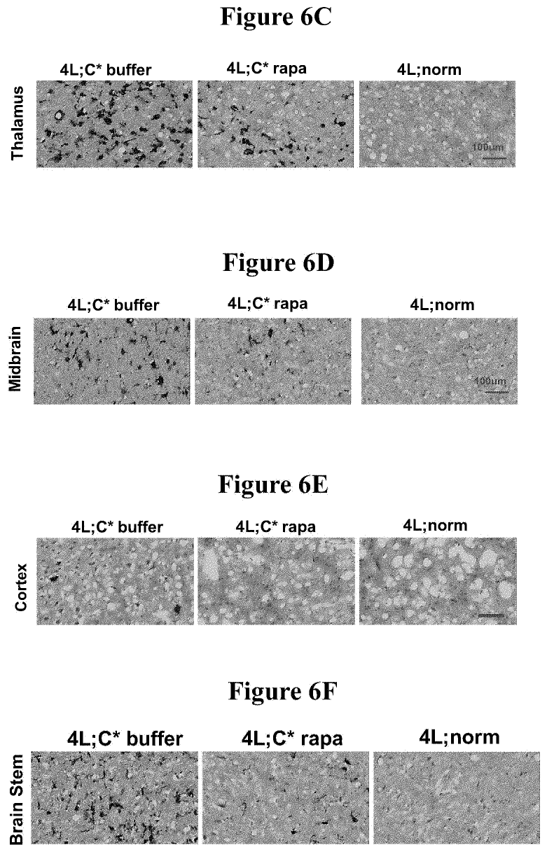

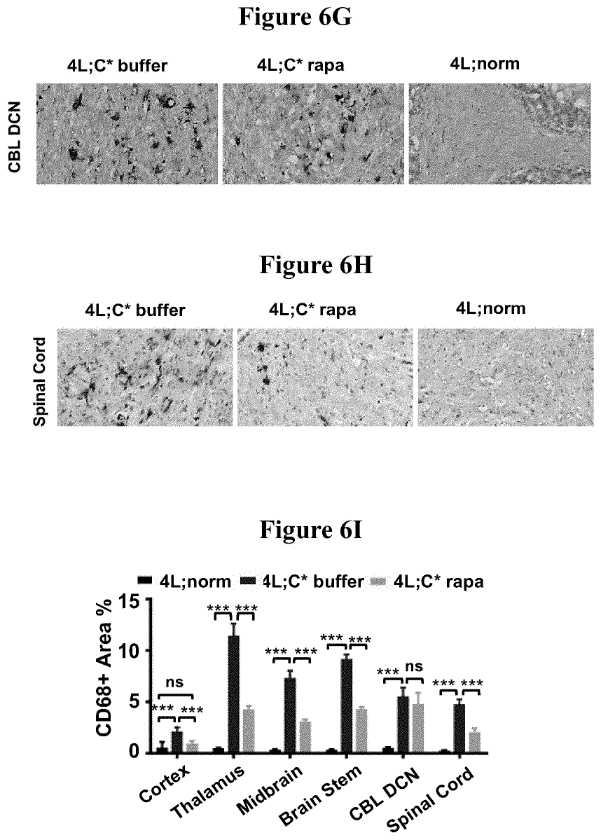

CD68, a classic marker of activated microglia/macrophage (Korzhevskii et al., (2016) Neurosci Behav Physi 46, 284-290), was used to determine CNS inflammation by immunohistochemistry staining. Using a similar immunohistochemistry staining method for CD68 detections that was used for GFAP staining described above, sagittal cryosections of brain harvested from rapamycin treated 4L;C* mice, buffer-treated 4L;C* mice, and 4L normal mice at age of Day 55 were analyzed by immunohistochemical staining with rat anti-mouse CD68 antibody for phagocytotic macrophage/activated microglia cells. FIG. 6A.

Thalamus showed the most intensive signals (brown) followed by brain stem, middle brain and cerebellar DCN regions, with minimal signal observed in cortex. Notably, rapamycin treatment significantly reduced CD68+ area in all brain regions (by 53-62%) in 4L;C* mice, except mild decrease in cerebellar DCN region. FIGS. 6B-6I. Compared to normal brain (4L;norm), 4L;C* buffer showed dramatically increased numbers of activated microglia/macrophage with enlarged cell body, while rapamycin treatment significantly decreased the number of activated macrophage/microglia and shrunk the cell body, especially in thalamus, middle brain and brain stem regions. FIGS. 6B-6I. For spinal cord, reduction of CD68 signals (by 56%) was also observed in 4L;C* rapamycin mice. FIGS. 6B and 6H.

Intensified CD68 signals were detected in most brain regions of 4L;C* buffer mice as compared to 4L normal controls. FIGS. 6A to 6I showed that thalamus exhibited the most intensive signals followed by brain stem, midbrain and cerebellar DCN regions, with moderate signal observed in cortex. Notably, rapamycin treatment significantly reduced CD68 signal area in all brain regions (by 53-62%) in 4L;C* mice, except mild decrease in cerebellar DCN region. FIG. 6G. Compared to normal brain (4L;norm), buffer-treated 4L;C* mouse brains showed dramatically increased numbers of activated microglia/macrophage with enlarged cell body, while rapamycin treatment significantly decreased these numbers of activated macrophage/microglia and shrunk the cell body in the 4L;C* mouse, especially in thalamus, middle brain and brain stem regions. FIGS. 6C, 6D, and 6F. For spinal cord reduction of CD68 signal (by 56%) was also observed in 4L;C* rapamycin mice. FIGS. 6B and 6H. These results revealed that rapamycin effectively ameliorates CNS inflammation both in brain and spinal cord.

Microglia, not only serve as primary immune cells in CNS, but can also regulate neuronal network and form bidirectional dynamic crosstalk with neuron. Astrocytes, activated by signals from injured neurons or activated microglia, are involved in maintenance and support of neurons, play direct roles in synaptic transmission. By releasing diverse signaling molecules, both microglia and astrocytes can establish autocrine feedback and bidirectional conversation for a tight reciprocal modulation. Thus microglia, astrocytes and neuron form an intimate cross talk network in CNS. Pronounced activation of microglia/macrophage and astrocyte in brain of nGD mice may be induced by, and contribute to the degeneration of neuron. The CD68 staining data suggest that both total and activated macrophage/microglia are decreased in nGD brain after rapamycin treatment (also supported by data in Example 6). Further, astrogliosis was also markedly reduced by rapamycin in nGD mice FIGS. 5A-5C. Significant decreased activation of glial cells may greatly benefit the function of neuron and improve the quality of life in nGD mouse model, considering their important role in neuron maintenance and regulation. Collectively, the data herein revealed that rapamycin significantly ameliorates neuropathological manifestations in nGD mice, which could contribute to prolonged survival and improved neurological deficits.

### Example 4. Hyperactivity of the Autophagosome-Lysosomal System and mTORC1 Pathway in nGD Brain were Normalized after Rapamycin Treatment

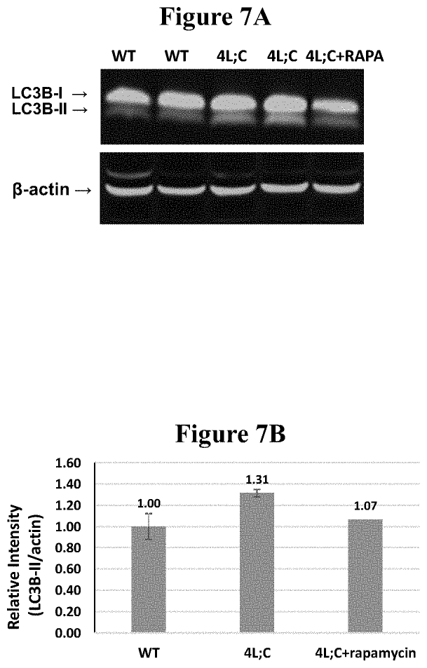

Like other lysosomal storage diseases, Gaucher disease encompasses a reduced ability of lysosomes to fuse with autophagosomes subsequently resulting in a defect in autophagosome maturation and defective degradation. To assess involvement of autophagy and the effect of rapamycin-treatment in Gaucher disease, midbrain tissue was harvested from well-perfused rapamycin-treated 4L;C* mice, buffer-treated 4L;C* mice, and normal C57BL/6J (WT) mice at age of Day 55. The harvested midbrain tissue was prepared for and used in Western blot analysis using standard techniques known in the field. Resulting immunoblots were probed for LC3B-II and β-actin. FIG. 7A. The lipid modified form of the microtubule-associated protein 1A/1B-light chain 3 (LC3), referred to as LC3B-II, associates with autophagosome membranes and was hence used as a marker for autophagy. Intensity of LC3B-II semi-quantified and normalized to β-actin for each sample. FIG. 7B. FIGS. 7A and 7B show that LC3B-II levels in the midbrain of buffer-treated 4L;C* mice were significantly higher than that of WT mice whereas LC3B-II levels in rapamycin-treated 4L;C* mice were comparable to those of WT mice. These data suggest that administration of rapamycin can normalize the abnormal increase of autophagosomes in a brain afflicted with Gaucher disease.

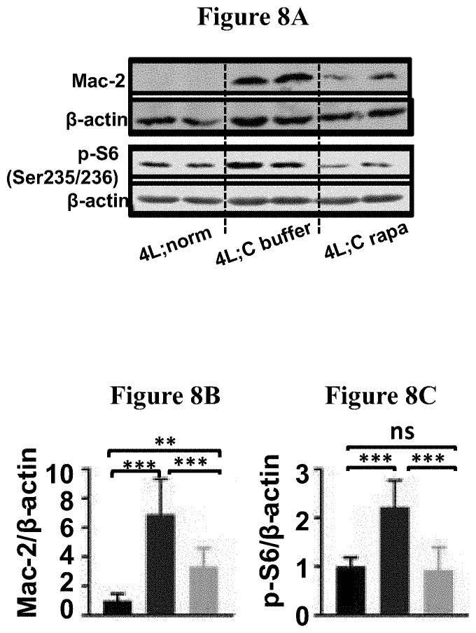

A regulator of autophagy-lysosomal function is the mTOR-dependent and mTOR-independent signaling pathways. Rapamycin is a classical inhibitor of mTORC1 and subsequently mTOR-dependent autophagy pathways. The potential change of mTORC1 signaling pathway in Gaucher disease and the effect of rapamycin-treatment on the pathway was assessed in midbrain tissue harvested from well-perfused rapamycin-treated 4L;C* mice, buffer-treated 4L;C* mice, and 4L normal mice (4L;norm) at age of Day 55. The harvested midbrain tissue was prepared for and used in Western blot analysis using standard techniques known in the field. Resulting immunoblots were probed for ribosome protein S6—an established downstream target of the mTORC1 signaling pathway. FIG. 8A. The phospho-S6 (Ser235/236) was upregulated in nGD brain (2.2-fold) and normalized after rapamycin treatment, suggesting mTORC1 pathway is hyperactive in nGD brain and normalized after rapamycin treatment. FIG. 8C. Immunoblot analysis was also performed for Mac 2, a marker for activated microglia/macrophages FIG. 8A. FIG. 8B shows that abnormally elevated Mac2 protein in the mouse nGD brain was significantly reduced by rapamycin treatment. Such reduction suggests either decreased activation of microglia cells or reduced number of macrophages after rapamycin treatment. The Mac2 data are consistent with the reduction of CD68+ signals demonstrated in immunohistochemistry analysis shown in FIGS. 6A-6I.

### Example 5. Rapamycin Administration Reduced Elevated Expression of Inflammatory Mediators in the Brain of a Neuronopathic Gaucher Disease (nGD) Murine Model

mRNA levels of certain inflammatory mediators were known to be significantly elevated in brains of nGD mouse models. Vitner et al., (2012) Brain. 135 (Pt 6):1724-35; Dasgupta et al., (2015) Hum Mol Genet. 15; 24(24):7031-48. The highly activated microglia/and astrocyte are the main source of multiple inflammatory mediators in brain. Sofroniew (2014), Cold Spring Harb Perspect Biol. 7(2):a020420. Considering the markedly decreased activation of glial cells in brain, it was hypothesized that the mRNA expression of these inflammatory mediators was decreased after rapamycin treatment. To test this hypothesis, frozen samples of midbrain tissue were collected from well-perfused rapamycin-treated 4L;C* mice, buffer-treated 4L;C* mice, and 4L normal, untreated mice at age of Day 55. mRNA was isolated from the frozen samples of midbrain tissue using methods standard in the field. The isolated mRNA was subjected to quantitative reverse transcription PCR (RT-qPCR) analysis using oligos directed toward inflammatory mediator genes of interest, including cytokine, chemokine, and inflammation-related receptors. Specifically, RT-qPCR analysis was performed to detect mRNA expression levels of the following genes: tumor necrosis factor (TNF)-alpha, interleukin (IL)-1-beta (IL-1β), IL-6, IL-17A, transforming growth factor beta (TGF-β), chemokine ligand (CCL) 5 (CCL5), CCL2, CCL22 chemokine (C-X-C motif) ligand 2 (CXCL2), intercellular adhesion molecule 1 (ICAM1), tumor necrosis factor receptor 1 (TNFR1), Cluster of Differentiation 86 (CD86), EGF-like module-containing mucin-like hormone receptor-like 1 (F4/80), component 5 (C5), and complement component 5a receptor 1 (C5AR1).

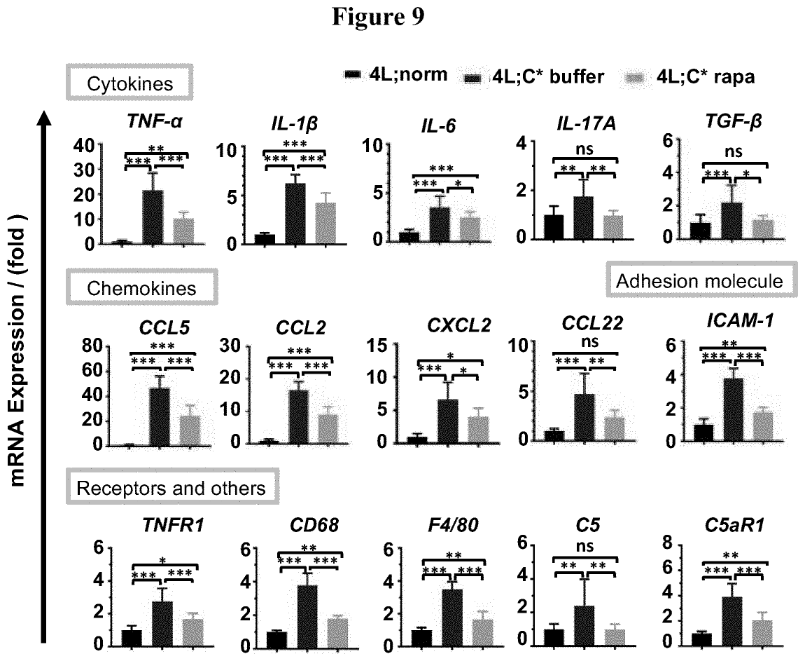

Tumor necrosis factor-α (TNF-α), a pro-inflammatory cytokine, which is produced primarily by activated microglia, macrophages, activated astrocytes and neurons, contributes to a variety of brain pathologies. FIG. 9 shows a dramatic increase (21.6 fold) of TNF-α and TNF receptors 1 (2.8 fold) in buffer-treated 4L;C* mice, which were significantly decreased in rapamycin-treated 4L;C* mice. The levels of proinflammatory cytokines IL-1β (6.2-fold) and IL-6 (4.2-fold) were also elevated in buffer-treated 4L;C* mice and reduced in rapamycin-treated 4L;C* mice. FIG. 9. Lymphocyte-derived pro-inflammatory cytokine IL-17A and immunosuppressive cytokine, transforming growth factor-β, were both increased in buffer-treated 4L;C* mice and normalized in rapamycin-treated 4L;C* mice. FIG. 9.

Chemokine are small heparin-binding proteins which direct the movement of circulating leukocytes to sites of inflammation or injury. The chemokine CCL5 was markedly elevated in buffer-treated 4L;C* mice (47-fold) compared to normal littermates and significantly reduced in rapamycin-treated 4L;C* mice (23-fold). FIG. 9. CCL2, a key regulator of monocytes and lymphocytes migration that mainly released by reactive astrocyte, endothelia, macrophage and microglia, was also significantly increased (by 16.6-fold) in buffer-treated 4L;C* mice and decreased in rapamycin-treated 4L;C* mice (9-fold). FIG. 9. The expression levels of chemokine CXCL2 (6.6-fold) and CCL22 (4.7-fold) were also elevated in buffer-treated 4L;C* mice, which were reduced or even normalized in rapamycin-treated 4L;C* mice. FIG. 9.

TNF-α has also been demonstrated to cause expression of pro-adhesive molecules on the endothelium. Here, the adhesion molecule ICAM-1, which is typically expressed on endothelial cells and mediates the transmigration of leukocytes, was increased (3.8-fold) in buffer-treated 4L;C* mice and decreased in rapamycin-treated 4L;C* mice. FIG. 9.

In agreement with the CD68 pathology staining shown in FIGS. 6A-6I, the mRNA expression of macrophage/microglia markers CD68 and F4/80 were elevated (by 3.8-fold and 3.5-fold) in buffer-treated 4L;C* mice and significantly reduced in rapamycin-treated 4L;C* mice. FIG. 9. Immunoblot analysis of another macrophage/microglia marker, Mac2, exhibited a similar change (FIG. 8B), suggesting a decreased activation or reduced number of microglia and/or macrophages after rapamycin treatment.

Also observed in buffer-treated 4L;C* mice was increased expression of complement C5 (precursor of C5a) and C5aR1, key components controlling inflammatory response in nGD mice. Both C5 and C5aR1 expression levels were significantly reduced in rapamycin-treated 4L;C* mice. FIG. 9.

Neuroinflammation, which is characterized by activation of glial cells (microglia and astrocytes) and release of proinflammatory cytokines/chemokines in CNS, has been implicated in a wide range of neurodegenerative disorders such as Alzheimer's and Parkinson's disease, as well as in some lysosomal storage diseases. Guzman-Martinez et al. (2019) Front Pharmacol. 10:1008. In nGD, microglia activation, astrogliosis and upregulation of pro-inflammatory mediators are also observed in mouse model and patients as shown here and by others. Burrow, et al., (2015) Mol. Genet. Metab., 114 (2), 233-241; Enquis et al., (2007) PNAS, 104 (44) 17483-17488; Vitner et al., (2012) Brain. 135 (Pt 6):1724-35. Microglia and astrocytes can establish autocrine feedback and bidirectional conversation for a tight reciprocal modulation by releasing diverse signaling molecules. Jha et al., (2019) The Neuroscientist, 25(3), 227-240. Collectively, the data in Example 5 showed that the abnormally elevated mRNA levels (or production) of proinflammatory cytokines, chemokines, adhesion molecules as well as other inflammatory mediators in untreated nGD were notably reduced in nGD brains after rapamycin treatment.

It was also observed herein that rapamycin significantly reduced the mRNA expression of proinflammatory mediators in 4L;C* mice, including cytokines, chemokines, adhesion molecule, complement factor and receptors. FIG. 9. Among the cytokines, TNF-α is known to activate microglia and astrocytes via TNF receptors, resulting in the expression of further pro-inflammatory and phagocytic genes, and is understood to be a physiological gliotransmitter concerned with synaptic regulation. However, high and sustained level of TNF-α can lead to neuronal damage. For example, TNF-α is a critical effector of dopaminergic neuron degeneration in Parkinson's disease and can induce neuronal death in glaucoma. Tezel, (2008) Prog Brain Res. 173:409-21. The significantly elevated expression of TNF-α and TNFR-1 in nGD mice shown in FIG. 9 herein may be a main effector for neurotoxicity and contribute one or more neurological deficits nGD brain, including those observed in FIGS. 2A, 2B, and 3A-3C herein. TNF-α and IL-1β can induce chemokine CCL5 production from both astrocytes and microglia in multiple sclerosis. CCL5 is chemotactic for leukocytes migration also regulates the function of microglia and astrocytes, as well as control the movement of calcium ions in neurons and modulate the glutamate release in nerve terminal. Pittaluga, (2017) Front Immunol. 8:1079. The increased expression of CCL5 in the nGD brain shown in FIG. 9 could affect its function on neuron, astrocyte and microglia, which may contribute to the brain pathology of nGD. Considering the important role of proinflammatory mediators in regulating function of glial and neuron cells as well as amplifying inflammatory response, the data in FIG. 9 suggest that the significant decrease of inflammatory mediators in the nGD brain after rapamycin treatment benefits the glial and neuron cell function and improves neurologic deficits.

### Example 6. Rapamycin Administration Suppressed Abnormal Proliferation and Activation of Resident Immune Cells (Microglia) and Reduced Leukocyte Migration into the Brain of a Neuronopathic Gaucher Disease (nGD) Murine Model

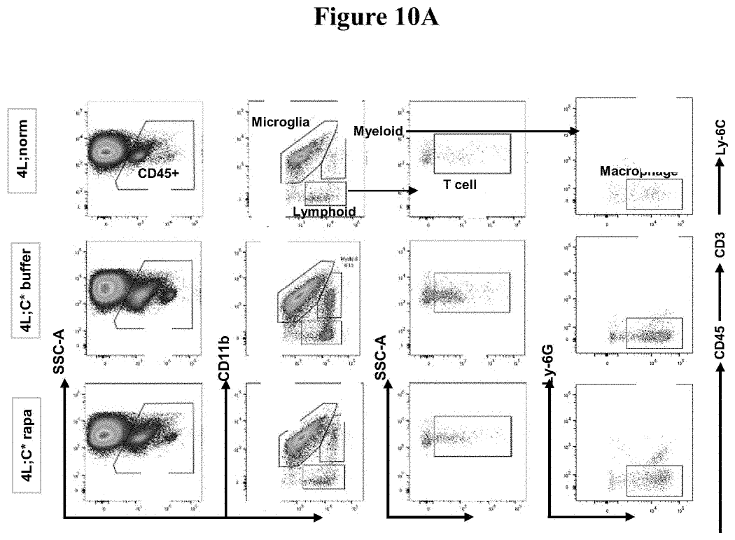

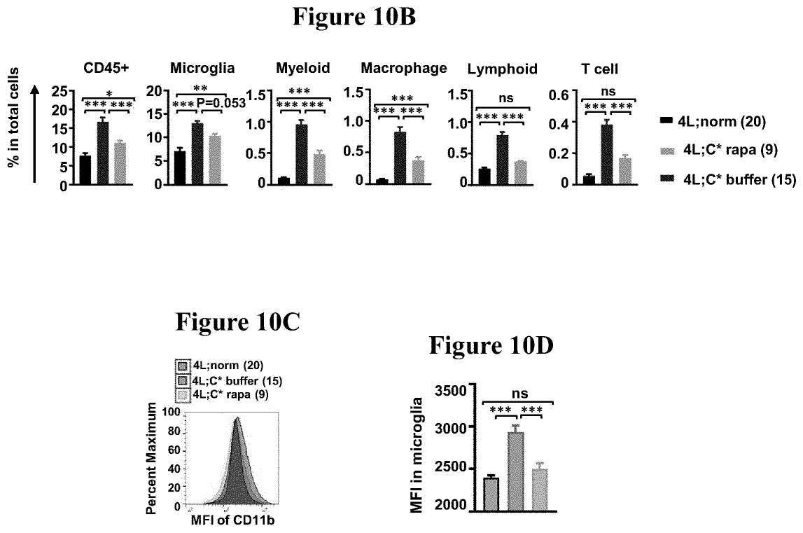

Brain inflammation invariably results in a reshaping of the blood-borne leukocyte populations inhabiting the CNS. To elucidate the changes of resident immune cells (i.e., microglial cells) and infiltrated leukocytes in nGD brain and the effect rapamycin has on activation of immune cells and infiltration, flow cytometry was used to analyze different leukocyte populations in brain parenchyma, including CD45⁺ cells, microglia, myeloid, lymphoid, macrophage and T cells. Briefly, the brain was harvested from well-perfused rapamycin-treated 4L;C* mice, buffer-treated 4L;C* mice, and 4L normal littermates at age of Day 50. The hemispheres of the brain were separated, minced, and digested by incubation with 200 mg/ml collagenase IV for 30 min at 37° C. The resulting cell suspensions were then fractionated by density gradient separation using 25% Percoll® gradients at 520×g for 20 minutes. Percoll®, a registered trademark of GE Healthcare, consists of colloidal silica particles of 15-30 nm diameter (23% w/w in water) which have been coated with polyvinylpyrrolidone (PVP) and is well suited for density gradient experiments because it possesses a low viscosity compared to alternatives, a low osmolarity, and no toxicity towards cells and their constituents. After removing myelin and top suspension layer, pelleted cells collected were then subjected to multicolor (5) fluorescence-activated cell sorting analysis (FACS) by co-staining with multiple antibodies that conjugated with various fluorochromes to identify microglia and subpopulations of leukocytes. FIG. 10A. The quantitative analysis of frequency of different leukocyte populations in the brain is provided in FIG. 10B. Data showed that the percentages of total CD45⁺ (including microglia and blood-borne leukocytes) was substantially increased (2.2-) in buffer-treated 4L;C* mice and significantly decreased (1.4-fold) in rapamycin-treated 4L;C* mice. FIG. 10B. The percentages of myeloid and macrophages showed the most significant increase (8.6-fold and 10.9-fold) in buffer-treated 4L;C* mice and both were markedly decreased in rapamycin-treated 4L;C* mice. FIG. 10B. Notably, both lymphoid and T cell population achieved normalization after rapamycin treatment. FIG. 10B.

The frequency of microglia (CD45^{intermediate}CD11b^{high}) was increased in buffer-treated 4L;C* mice (1.9 fold) and decreased in rapamycin-treated 4L;C* mice (1.3-fold), which indicates reduced microgliosis in nGD brain. FIG. 10B. To assess if activation status of microglia was reduced in nGD brain, the mean fluorescence intensity (MFI) of CD11b, a marker upregulated in activated microglia, was measured. Data showed that CD11b was increased in buffer-treated 4L;C* mice and in rapamycin-treated 4L;C* mice, suggesting reduced microglia activation. FIGS. 10C and 10D.

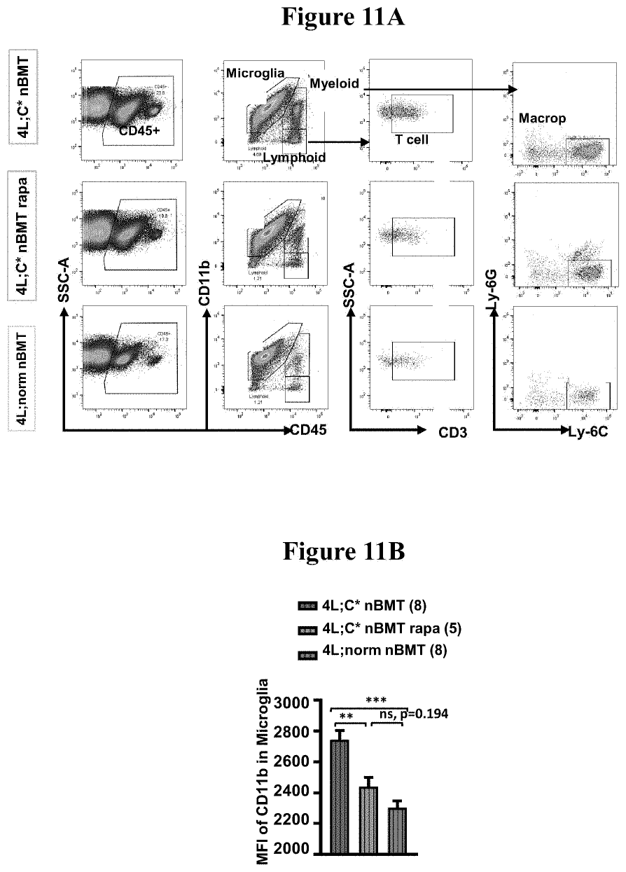

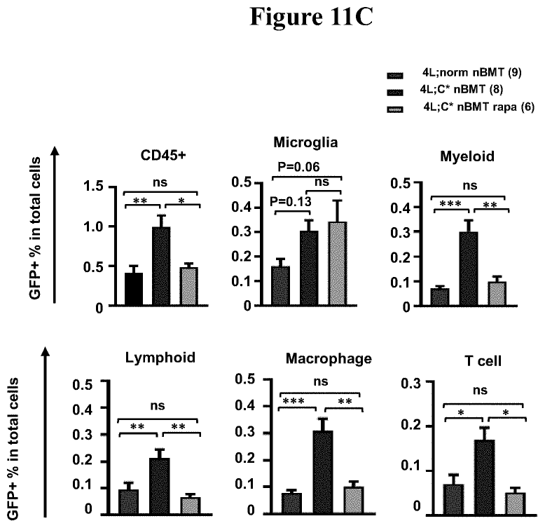

In order to determine if rapamycin treatment indeed reduced leukocyte migration, in addition to downregulated resident microglia activation, tracking of migrating cells was assessed. Briefly, mice that express enhanced green fluorescent protein (GFP; C57BL/6 GFP/CD45.1) were used as bone marrow donors. Low-density bone marrow (LDBM) cells were harvested by flushing the hind-leg bones with Dulbecco's modified Eagle's medium (DMEM) containing 2% fetal calf serum (FCS) and 50 U penicillin/0.05 mg/mL streptomycin using a 23 G needle. The cells were passed through a 100 μm cell strainer. LDBM cells were isolated after density gradient centrifugation with Histopaque-1083. Viable cells were counted with trypan blue dye and resuspended in PBS containing 30 U/mL heparin. The recipient pups (˜3 days old of 4L;C or 4L;norm) were subjected to myelosuppressive conditioning with the chemotherapeutic agent busulfan by intraperitoneal injection at 20 mg/kg. Twenty-four hours later, the 4L;C and 4L;norm recipient pups then received about 1×10⁶ GPF+ donor cells via injection into super-facial temple vein (nBMT). 4L;C nBMT mice were then given an i.p. injection of rapamycin or buffer every other days starting at 21 days of age. Once the animals reach 55 days of age, brains were harvested from well-perfused rapamycin-treated 4L;C* nBMT mice, buffer-treated 4L;C* nBMT mice, and 4L nBMT normal mice, hemispheres were minced into a cell suspension that is subsequently applied to Percoll density-gradient isolation as described above. Multicolor FACS analysis was conducted as described above to analyze contribution of GFP+ migrated cells in microglia and subpopulations of leukocytes. FIG. 11A. The frequency of donor-derived GFP+ cells among different leukocyte populations in brain parenchyma was quantified, including CD45⁺ cells, microglia, myeloid, lymphoid, macrophage and T cells. Table 1. In busulfan-conditioned mice, engraftment of GFP+ microglial cells was increased in 4L;C* mice compared to 4L normal mice, regardless of treatment with buffer or rapamycin. Table 1. However, the CD11b intensity of overall microglia population increased in buffer-treated 4L;C*nBMT mice was normalized in rapamycin-treated 4L;C* mice, suggesting downregulation of microglia activation by rapamycin treatment. FIG. 11B.

The average composition of GFP⁺ cells in peripheral blood leukocytes achieved higher than 96% in all nBMT groups (data not shown). FIG. 11C shows that buffer-treated 4L;C* nBMT mice had an increased amounts of GFP+ donor-derived CD45⁺ cells, myeloid, lymphoid, macrophage and T cell populations whereas increased amount of GFP+ migrated cells from these same populations was all normalized (similar to that of 4L;norm nBMT mice) in rapamycin-treated 4L;C* nBMT mice. The data suggest rapamycin treatment can reduce leukocyte migration into the brain.

TABLE 1
Frequency of GFP+ Cells Among Various Leukocyte Populations

| | | | | Macro- | | T |
|---|---|---|---|---|---|---|
| | CD45+ | Microglia | Myeloid | phage | Lymphoid | Cell |
| 4L; C* | 4.92 | 1.71 | 23.0 | 24.7 | 26.8 | 31.9 |
| nBMT | | | | | | |
| buffer | | | | | | |
| 4L; C* | 2.87 | 1.56 | 17.4 | 19.5 | 29.3 | 37.6 |
| nBMT | | | | | | |
| rapamycin | | | | | | |
| 4L; norm | 1.96 | 0.73 | 27.3 | 37.3 | 40.7 | 47.3 |
| nBMT | | | | | | |
| untreated | | | | | | |

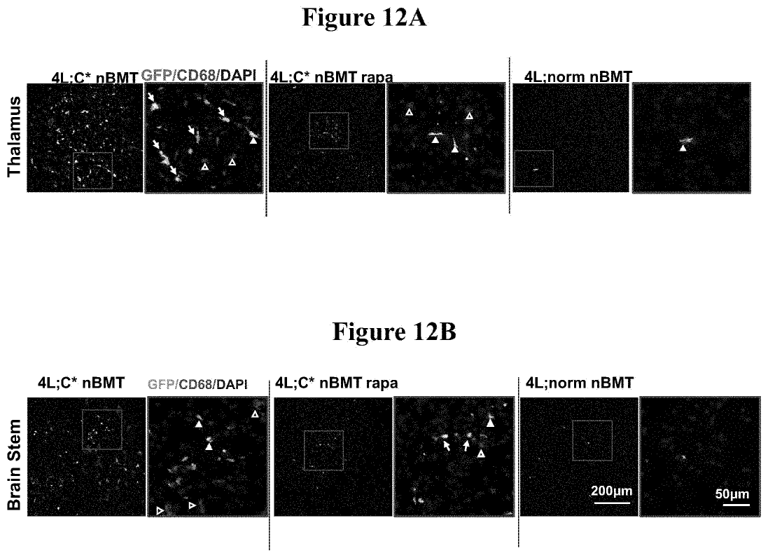

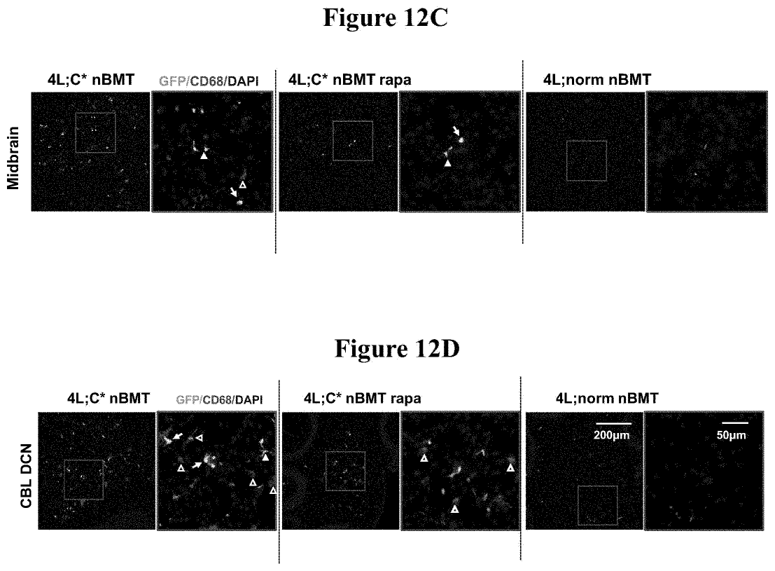

The reduced migration of leukocytes into brain was further validated by immunofluorescence staining using methods similar to that described above. Buffer-treated 4L;C* nBMT mice exhibited intensive GFP⁺ migrated cells in thalamus region, followed by brain stem, midbrain and cerebellar DCN. FIGS. 12A-12D. In rapamycin-treated 4L;C* nBMT mice, the GFP⁺ cells were dramatically reduced in thalamus region, and mildly decreased in brain stem, midbrain and cerebellar DCN region. By co-staining for macrophage/activated microglia with the marker CD68, it was found that both migrated macrophages (GFP⁺CD68⁺) and activated microglia (CD68⁺ only) were decreased across the brain after rapamycin treatment (rapamycin-treated 4L;C* nBMT mice).

The data show significant increased infiltration of leukocytes, especially macrophages and T cells in nGD brain, which could be recruited/facilitated by the increased adhesion molecular ICAM-1 and chemokines such as CCL5 and CCL2 (FIG. 9), two main regulators of leukocytes migration. The data also show that the migration of leukocytes, especially macrophage and T cells were dramatically reduced and even normalized in the 4L;C* nBMT model by rapamycin treatment. The reduced migration of leukocytes into the brain, decreases the expression and release of proinflammatory mediators in nGD brain, and vice versa, decreased inflammatory level also results in reduction of infiltrated leukocytes into brain.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method of improving at least one neurological function in a subject, the method comprising: administering to a subject in need thereof an effective amount of a rapamycin compound, wherein the subject has or is suspected of having a neurologic lysosomal storage disorder.

2. The method of claim 1, wherein the rapamycin compound is sirolimus, everolimus, temsirolimus, ridaforolimus, N-dimethylglycinate-rapamycin, 32-deoxo-rapamycin, zotarolimus, acrolimus or pimecrolimus.

3. The method of claim 1, wherein the rapamycin compound is conjugated to a pharmaceutically acceptable polymer.

4. The method of claim 1, wherein the rapamycin compound is formulated in a pharmaceutical composition, which further comprises a pharmaceutically acceptable carrier.

5. The method of claim 1, wherein the rapamycin compound is administered to the subject by a parenteral route or orally.

6. The method of claim 1, wherein the neurologic lysosomal storage disease is selected from the group consisting of Fabry disease, Farber disease, Gangliosidosis GM1, Krabbe disease, Schindler disease, Sandhoff disease, Tay-Sachs, Metachromatic Leukodystrophy, Niemann-Pick disease, Hurler syndrome, Hurler-Scheie syndrome, Hunter syndrome, Sanfilippo A syndrome, Sanfilippo B syndrome, Sanfilippo C syndrome, Sanfilippo D syndrome, Sly Syndrome, Pompe disease, and Gaucher disease.

7. The method of claim 6, wherein the neurologic lysosomal storage disorder is neuronopathic Gaucher disease (nGD).

8. The method of claim 1, wherein the subject is a human patient having the neurologic lysosomal storage disorder.

9. The method of claim 8, wherein the subject is a human patient having Type II or Type III nGD.

10. The method of claim 1, wherein the subject is a human child patient having the neurologic lysosomal disorder.

11. The method of claim 1, wherein the subject has undergone or is undergoing another therapy for the neurologic lysosomal disorder.

12. The method of claim 1, wherein the rapamycin compound is administered at a dose that leads to an about 5 to about 60 ng/ml of the rapamycin compound in the serum of the subject.

13. The method of claim 1, wherein the rapamycin compound is administered by a schedule ranging from three times per day to once per week.

14. The method of claim 1, wherein the rapamycin compound is administered once a day orally or once a day to once a week by intravenous infusion.

15. A method of treating a neurologic lysosomal storage disease in a subject, the method comprising: administering to a subject in need thereof an effective amount of a rapamycin compound.

16. The method of claim 15, wherein the rapamycin compound is sirolimus, everolimus, temsirolimus, ridaforolimus, N-dimethylglycinate-rapamycin, 32-deoxo-rapamycin, zotarolimus, acrolimus or pimecrolimus.

17. The method of claim 15, wherein the rapamycin compound is conjugated to a pharmaceutically acceptable polymer.

18. The method of claim 15, wherein the rapamycin compound is formulated in a pharmaceutical composition, which further comprises a pharmaceutically acceptable carrier.

19. The method of claim 15, wherein the rapamycin compound is administered to the subject by a parenteral route or orally.

20. The method of claim 15, wherein the neurologic lysosomal storage disease is selected from the group consisting of Fabry disease, Farber disease, Gangliosidosis GM1, Krabbe disease, Schindler disease, Sandhoff disease, Tay-Sachs, Metachromatic Leukodystrophy, Niemann-Pick disease, Hurler syndrome, Hurler-Scheie syndrome, Hunter syndrome, Sanfilippo A syndrome, Sanfilippo B syndrome, Sanfilippo C syndrome, Sanfilippo D syndrome, Sly Syndrome, Pompe disease, and Gaucher disease.

21. The method of claim 20, wherein the neurologic lysosomal storage disorder is neuronopathic Gaucher disease (nGD).

22. The method of claim 15, wherein the subject is a human patient having the neurologic lysosomal storage disorder.

23. The method of claim 22, wherein the subject is a human patient having Type II or Type III nGD.

24. The method of claim 15, wherein the subject is a human child patient having the neurologic lysosomal disorder.

25. The method of claim 15, wherein the subject has undergone or is undergoing another therapy for the neurologic lysosomal disorder.

26. The method of claim 15, wherein the rapamycin compound is administered at a dose that leads to an about 5 to about 60 ng/ml of the rapamycin compound in the serum of the subject.

27. The method of claim 15, wherein the rapamycin compound is administered by a schedule ranging from three times per day to once per week.

28. The method of claim 15, wherein the rapamycin compound is administered once per day orally or once per day to once per week by intravenous infusion.
